(19) United States
(12) Patent Application Publication
(10) Pub. No.: US 2026/0094685 A1
(43) Pub. Date: Apr. 2, 2026

(54) METHOD AND SYSTEM FOR RECOMMENDING AI-BASED SOLUTIONS FOR INFANT AND TODDLER HEALTH CARE

(71) Applicant: Ddotcare Co., Ltd, Gwangju (KR)

(72) Inventors: Su Jin HUH, Yongin-si (KR); Eun Kyoung BOM, Jeollanam-do (KR); Chang Ha LEE, Yongin-si (KR); Jae Young SONG, Seoul (KR); Dong Ye KIM, Gwangju (KR)

(21) Appl. No.: 18/930,880

(22) Filed: Oct. 29, 2024

(30) Foreign Application Priority Data
Sep. 30, 2024 (KR) 10-2024-0132892

(51) Int. Cl. G16H 20/10; G16H 10/60

(52) U.S. Cl. CPC G16H 20/10; G16H 10/60

(57) ABSTRACT

A method for recommending an artificial intelligence (AI)-based solution for infant and toddler healthcare, comprising: collecting life pattern data, including feeding or sleep data, via a data collection unit; refining the collected life pattern data and developmental data to extract a plurality of monthly age life patterns by matching the refined data, through a life pattern extraction unit; analyzing the extracted monthly age life patterns to identify a problem and corresponding correlation factors for each pattern using a correlation factor extraction unit; measuring a similarity between actual input data and the extracted life patterns via a similarity measurement unit; inputting the actual data and the extracted patterns into an AI neural network model to perform learning and generate correlation factor weights through a learning unit; and recommending a non-prescription medication based on symptom analysis performed by a large language model (LLM) through a recommendation unit.

### TECHNICAL FIELD

The present disclosure relates to a method and system for recommending an artificial intelligence (AI)-based solution for infant and toddler health care, and relates to a method and system for recommending an AI-based solution for infant and toddler health care that can recommend a customized life pattern schedule for each infant and toddler based on a simple questionnaire.

### BACKGROUND

Sleep is a factor that greatly affects growth during an infant and toddler period. In particular, research results illustrate that sleep has a great impact on obesity rates, growth hormone secretion, or the like. Therefore, securing an appropriate amount of sleep time during the infant and toddler period is important, and for this purpose, accurate information on when to feed and when to take a nap is requested.

However, in the case of infants, there are very large changes in patterns such as an amount of feeding, an interval between feedings, an amount of sleep, and an amount of sleep time every few weeks after birth. In other words, recommendations for infants to develop properly and have stable sleep change greatly. For example, a recommended feeding amount and recommended sleep time may vary depending on how much the infant was fed the previous day and how much the infant slept the previous day, and the recommended feeding amount and recommended sleep time may vary greatly depending on whether the infant is currently 3 weeks old or 6 weeks old. These recommended feeding amounts and recommended sleep times cannot be accurately adjusted without the help of a professional.

As the related art, there is Korean Patent No. 10-0659695 (system and method for providing service to check growth and development stage of infant), but this only discloses a technology for checking the growth and development stages of infants by performing statistical processing together with existing standard growth and development data on infant growth and development to produce statistical data, and then performing a comparative analysis of the growth and development status of infants based on the calculated statistical data at a request of a client.

### SUMMARY

An object of the present disclosure is to provide a method and system for recommending an AI-based solution for infant and toddler health care capable of providing a customized life pattern schedule that includes optimal sleep time and feeding time for each infant and toddler based on only basic information about the infant and toddler and the results of a simple questionnaire.

According to an aspect of the present disclosure, there is provided a method for recommending an artificial intelligence (AI)-based solution for infant and toddler health care, the method including: collecting life pattern data including feeding data or sleep data of an infant and toddler, by a data collection unit; refining development data and life pattern data from the collected life pattern data and extracting a plurality of monthly age life patterns by matching the refined life pattern data and development data, by a life pattern extraction unit; analyzing the plurality of extracted monthly age life patterns to extract a problem and life pattern correlation factor for each monthly age life pattern, by a correlation factor extraction unit; measuring a similarity between actual input data and the monthly age life pattern, by a similarity measurement unit; inputting the actual input data and monthly age life patterns into an AI neural network model to perform learning, and deriving weight for each correlation factor, by a learning unit; extracting a question about a correlation factor having the highest weight from a database and providing the extracted question to a user terminal, by a questionnaire; and generating a customized life pattern schedule based on a questionnaire result input from the user terminal and providing the generated customized life pattern schedule to the user terminal, by the recommendation unit.

According to the present disclosure, it is possible to easily provide the customized life pattern schedule for each infant and toddler through a simple questionnaire without directly inputting the feeding data and sleep data of infants and toddlers.

In addition, based on a feeding amount or feeding time, the recommended feeding amount or recommended feeding time is automatically calculated and provided to the user in real time, and thus, the feeding amount or feeding time for infants and toddlers can be notified in a timely manner to help the feeding.

### BRIEF DESCRIPTION OF THE DRAWINGS

FIG. 1 is a flow chart explaining a method for recommending an AI-based solution for infant and toddler health care according to one embodiment of the present disclosure.

FIG. 2 is a configuration diagram explaining a system for recommending an AI-based solution for infant and toddler health care according to one embodiment of the present disclosure.

FIGS. 3 and 4 are diagrams explaining a determination method using cosine similarity according to one embodiment of the present disclosure.

FIGS. 5A, 5B, 6A, 6B, 7A, 7B, 8A, 8B, 9A, 9B, 9C are diagrams explaining application service operation screens according to one embodiment of the present disclosure.

### DETAILED DESCRIPTION

A specific structural or functional description of embodiments according to the concept of the present disclosure disclosed in the present specification is merely exemplified for the purpose of explaining embodiments according to the concept of the present disclosure, and embodiments according to the concept of the present disclosure may be implemented in various forms and are not limited to the embodiments described in the present specification.

Since embodiments according to the concept of the present disclosure may have various changes and may have various forms, embodiments are illustrated in the drawings and described in detail in the present specification. However, these are not intended to limit embodiments according to the concept of the present disclosure to specific disclosed forms, and include all modifications, equivalents, or substitutes included in the spirit and technical scope of the present disclosure.

The terms used in the present specification are only used to describe specific embodiments and are not intended to limit the present disclosure. The singular expression includes the plural expression unless the context clearly indicates otherwise. In the present specification, the terms “include”, “have”, and the like are intended to specify the presence of a feature, number, step, operation, component, part, or combination thereof described in the present specification, but should be understood as not excluding in advance the possibility of the presence or addition of one or more other features, numbers, steps, operations, components, parts, or combinations thereof.

Hereinafter, embodiments of the present disclosure will be described in detail with reference to the drawings attached to the present specification.

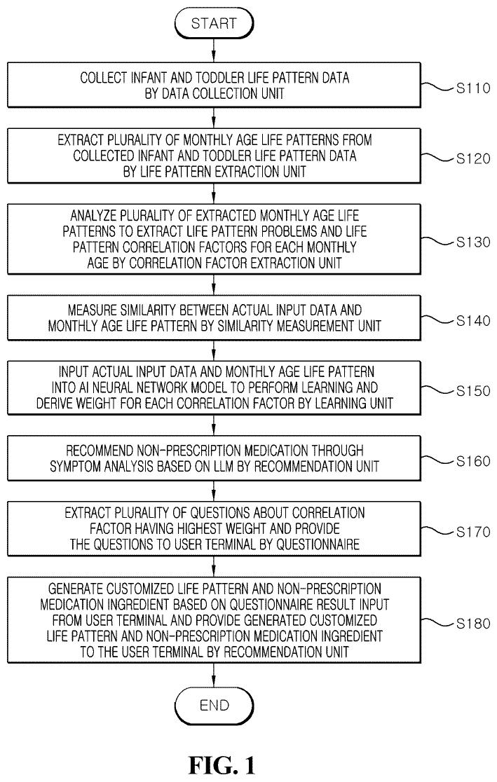

FIG. 1 is a flow chart explaining a method for recommending an AI-based solution for infant and toddler health care according to one embodiment of the present disclosure.

Referring to FIG. 1, in the method for recommending an AI-based solution for infant and toddler health care of the present disclosure, first, a data collection unit 110 collects life pattern data including feeding data or sleeping data of an infant and toddler (S110). At this time, the feeding data includes the number of times and intervals of feeding during the day and the number of times and intervals of feeding at night, and the sleeping data includes the number of naps, total nap time, total sleep time, nighttime sleep start time, nighttime sleep end time, duration, and previous sleep interval. The life pattern data may include weaning food data, and weaning food data may include the number of times of weaning food and the interval between weaning food.

A life pattern extraction unit 120 may refine development data and life pattern data from the collected life pattern data, and extract a plurality of monthly age life patterns by matching the refined life pattern data and development data (S120). The life pattern extraction unit 120 may organize the extracted life pattern data into similar patterns, and refine the data by deleting patterns that are determined to be special cases that cannot be recommended among the organized similar patterns. The life pattern extraction unit 120 may extract the monthly age life patterns through the above-mentioned refinement process. For example, the following monthly age life patterns may be extracted. The life pattern extraction unit 120 may extract the plurality of monthly age life patterns.

A correlation factor extraction unit 130 may analyze the plurality of extracted monthly age life patterns to extract a problem and a life pattern correlation factor for each monthly age life pattern (S130). First, the correlation factor extraction unit 130 may extract the problem for each monthly age life pattern, organize the correlation factor used to determine the problem, and organize an occurrence frequency of the problem by monthly age.

The correlation factor extraction unit 130 may extract correlation factors centered on overlapping correlation factors among correlation factors related to problems by extracted monthly age life pattern and correlation factors related to life patterns (S130). The correlation factor extraction unit 130 may determine nine correlation factors such as the number of naps, total nap time, total nighttime sleep time, total sleep time, total number of feedings, feeding period, weaning period, number of times of weaning, and number of nighttime feedings as major related factors, and match the plurality of life patterns extracted from the life pattern extraction unit with the problems.

The similarity measurement unit 140 may measure a similarity between actual input data and the monthly age life pattern (S140). The similarity measurement unit 140 may convert a time of each column among infant and toddler standard life patterns by monthly age into a number table in decimal units and scale the time. The similarity measurement unit 140 may also scale the actual input data of a user in the same way. The similarity measurement unit 140 may set the variables of the infant and toddler standard life pattern by a monthly age to A1 to A9, set the variables of the actual input data of the user to B1 to B9, and calculate the cosine similarity by applying a separate scale control table. At this time, the scale control table may set weights to C1 to C9, and the C1 to C9 variables may be weights based on expert knowledge and frequency of occurrence of major problems by the monthly age. The similarity measurement unit 140 may measure similarity according to similarity calculation formulas below.

$$\begin{matrix}AA = SQRT [ {( A 1 * C 1 )}^{2} + {( A 2 * C 2 )}^{2} + … + {( A 9 * C 9 )}^{2} ] \\ BB = SQRT [ {( B 1 * C 1 )}^{2} + {( B 2 * C 2 )}^{2} + … + {( b 9 * C 9 )}^{2} ] \\ MM = [ ( A 1 * C 1 * B 1 * C 1 ) + ( A 2 * C 2 * B 2 * C 2 ) + … + ( A 9 * C 9 * B 9 * C 9 ) ] \\ Similarity = AA * BB / MM\end{matrix}$$

The variables A1 to A9 of the infants and toddler standard life patterns by monthly age and the variables B1 to B9 of the actual input data of the user may be nine correlation factors such as the number of naps, total nap time, total nighttime sleep time, total sleep time, total number of feedings, feeding period, weaning period, number of times of weaning, and number of nighttime feedings.

A learning unit 150 may input the actual input data and monthly age life pattern into an AI neural network model to perform learning, and derive weights for each correlation factor (S150). The learning unit 150 may input the C1 to C9 variable values corresponding to weights into the neural network model to perform learning. In this case, learning may be performed between the actual input data of the user and a suggested life schedule of an expert, and optimal weights for each monthly age and problem may be derived.

A recommendation unit may recommend non-prescription medication through symptom analysis based on a large language model (LLM) (S160).

A questionnaire 160 may extract a question about a correlation factor having the highest weight from a questionnaire DB 170 and provide the question to the user terminal 200 (S170). The questionnaire 160 may extract the life pattern schedule of the user through general questions including the monthly age of the user, the problem to be solved, or the like, and provide the user with a customized life pattern schedule without data input through a question about the correlation factor having the highest weight derived from the artificial intelligence learning model. The questionnaire 160 may extract the question about the correlation factor having the highest weight among a plurality of questions stored in the questionnaire DB 170 and provide the extracted question to the user.

A recommendation unit 180 may generate a customized life pattern and non-prescription medication ingredient based on the questionnaire result input from the user terminal 200 and provide the generated customized life pattern and non-prescription medication ingredient to the user terminal (S180).

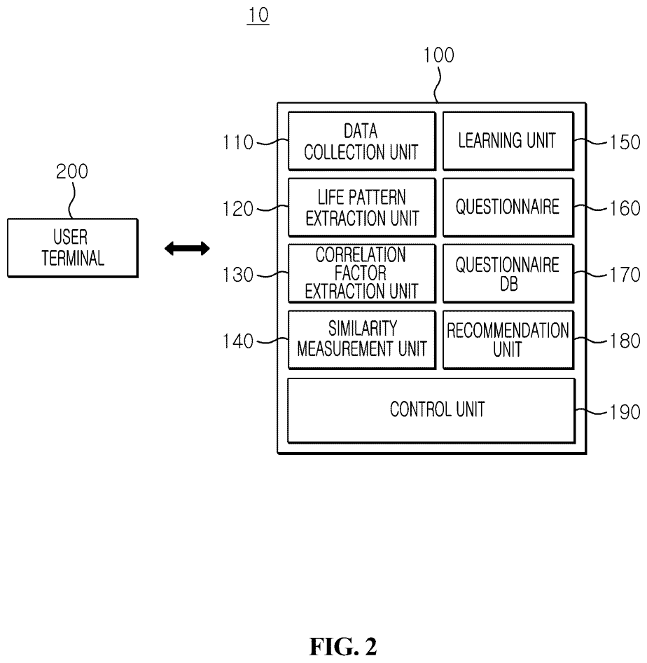

FIG. 2 is a configuration diagram explaining a system for recommending an AI-based solution for infant and toddler health care according to one embodiment of the present disclosure. The configuration of a system 10 illustrated in FIG. 2 is only a simplified example. In one embodiment of the present disclosure, the operation server and the user terminal of the system may include other configurations for performing a computing environment, and only some of the disclosed configurations may constitute the system 10.

Referring to FIG. 2, the system 10 for recommending an AI-based solution for infant and toddler health care may include an operation server 100 and a user terminal 200. The operation server 100 may include the data collection unit 110, the life pattern extraction unit 120, the correlation factor extraction unit 130, the similarity measurement unit 140, the learning unit 150, the questionnaire 160, the questionnaire DB 170, the recommendation unit 180, and the control unit 190.

The data collection unit 110 may collect the life pattern data including the feeding data or sleeping data of infants and toddlers. The data collection unit 110 may collect the feeding data or sleeping data of infants and toddlers directly input by users of the application service, and may collect them through sentence-type data such as memos input by users. The feeding data includes the number of times and intervals of feeding during the day and the number of times and intervals of feeding at night, and the sleeping data includes the number of naps, total nap time, total sleep time, nighttime sleep start time, nighttime sleep end time, duration, and previous sleep interval. The life pattern data may include weaning food data, and weaning food data may include the number of times of weaning food and the interval between weaning food.

The life pattern extraction unit 120 may refine development data and life pattern data from the collected life pattern data, and extract a plurality of monthly age life patterns by matching the refined life pattern data and development data. The life pattern extraction unit 120 may organize the extracted life pattern data into similar patterns, and refine the data by deleting patterns that are determined to be special cases that cannot be recommended among the organized similar patterns. The life pattern extraction unit 120 may extract the monthly age life patterns through the above-mentioned refinement process.

The correlation factor extraction unit 130 may extract correlation factors centered on overlapping correlation factors among correlation factors related to problems by extracted monthly age life pattern and correlation factors related to life patterns. The correlation factor extraction unit 130 may determine nine correlation factors such as the number of naps, total nap time, total nighttime sleep time, total sleep time, total number of feedings, feeding period, weaning period, number of times of weaning, and number of nighttime feedings as major related factors, and match the plurality of life patterns extracted from the life pattern extraction unit with the problems. In the present disclosure, nine correlation factors are confirmed as main related factors and described, but there is no limitation on the number of correlation factors.

The similarity measurement unit 140 may measure the similarity between the actual input data for infants and toddlers and the monthly age life patterns. The actual input data for infants and toddlers may include the feeding data or sleeping data. The similarity measurement unit 140 may convert a time of each column among the infant and toddler standard life patterns by monthly age into a number table in decimal units and scale the time. The similarity measurement unit 140 may also scale the actual input data of a user in the same way. The similarity measurement unit 140 may set the variables of the infant and toddler standard life pattern by a monthly age to A1 to A9, set the variables of the actual input data of the user to B1 to B9, and calculate the cosine similarity by applying a separate scale control table. In this case, the scale control table may set the weights to C1 to C9, and the C1 to C9 variables may be weights based on expert knowledge and frequency of occurrence of major problems by the monthly age. The similarity measurement unit 140 can measure similarity according to the similarity calculation formulas below.

$$\begin{matrix}AA = SQRT [ {( A 1 * C 1 )}^{2} + {( A 2 * C 2 )}^{2} + … + {( A 9 * C 9 )}^{2} ] \\ BB = SQRT [ {( B 1 * C 1 )}^{2} + {( B 2 * C 2 )}^{2} + … + {( b 9 * C 9 )}^{2} ] \\ MM = [ ( A 1 * C 1 * B 1 * C 1 ) + ( A 2 * C 2 * B 2 * C 2 ) + … + ( A 9 * C 9 * B 9 * C 9 ) ] \\ Similarity = AA * BB / MM\end{matrix}$$

The learning unit 150 may input the actual input data and monthly age life pattern into the AI neural network model to perform learning, and derive weights for each correlation factor. The learning unit 150 can input variable values of C1 to C9 corresponding to weights into the neural network model to perform learning. In this case, learning may be performed between the actual input data of the user and the suggested life schedule of the expert, and the optimal weights for each month and problem may be derived.

In the present specification, neural network and network function may be used interchangeably. The neural network may include a set of interconnected computational units, which can be generally referred to as nodes. These nodes may also be referred to as neurons. The neural network includes at least one node. The nodes (or neurons) that make up the neural network may be interconnected by one or more links. Within the neural network, one or more nodes connected through the links may relatively form an input node and output node relationship. The concepts of input node and output node are relative, and any node in the output node relationship with one node may be in the input node relationship with another node, and vice versa. As described above, the relationship of input node to output node may be created around the links. One or more output nodes may be connected to one input node through the links, and vice versa.

In the relationship between the input node and the output node connected through one link, the value of data of the output node may be determined based on data input to the input node. In this case, the link connecting the input node and the output node may have a weight. The weight may be variable and may be varied by a user or algorithm in order for the neural network to perform the desired function. For example, when one or more input nodes are connected to one output node by respective links, the output node may determine the output node value based on the values input to the input nodes connected to the output node and the weights set for the links corresponding to the respective input nodes.

As described above, in the neural network, one or more nodes are interconnected through one or more links to form the input node and output node relationship within the neural network. The characteristics of the neural network may be determined according to the number of nodes and links, the correlation between the nodes and the links, and the value of the weight assigned to each link within the neural network. For example, when there are two neural networks with the same number of nodes and links and different weight values of the links, the two neural networks may be recognized as different from each other.

The neural network may include a set of one or more nodes. A subset of nodes that constitutes the neural network may form a layer. Some of the nodes constituting the neural network may form one layer based on the distances from the initial input node. For example, a set of nodes with a distance n from the initial input node may constitute an n layer. The distance from the initial input node may be defined by the minimum number of links that should be passed to reach the node from the initial input node. However, this definition of the layer is arbitrary for explanation purposes, and the order of the layer within the neural network may be defined in a different way than described above. For example, a layer of nodes may be defined by the distance from the final output node.

The initial input node may refer to one or more nodes in the neural network through which data is directly input without going through any link in relationships with other nodes. Alternatively, in the neural network, in the relationship between nodes based on links, the initial input node may mean nodes that do not have other input nodes connected by links. Similarly, the final output node may refer to one or more nodes that do not have an output node in relationship with other nodes among the nodes in the neural network. In addition, hidden nodes may refer to nodes constituting the neural network other than the initial input node and the final output node.

The neural network according to one embodiment of the present disclosure may be a neural network in which the number of nodes in the input layer may be the same as the number of nodes in the output layer, and the number of nodes decreases and then increases as it progresses from the input layer to the hidden layer. In addition, the neural network according to another embodiment of the present disclosure may be a neural network in which the number of nodes in the input layer may be less than the number of nodes in the output layer, and the number of nodes decreases as it progresses from the input layer to the hidden layer. Further, the neural network according to still another embodiment of the present disclosure may be a neural network in which the number of nodes in the input layer may be greater than the number of nodes in the output layer, and the number of nodes increases as it progresses from the input layer to the hidden layer. The neural network according to still another embodiment of the present disclosure may be a neural network that is a combination of the above-described neural networks.

A deep neural network (DNN) may refer to a neural network that includes multiple hidden layers in addition to the input layer and output layer. The deep neural network can be used to identify latent structures in data. In other words, it is possible to identify the latent structure of a photo, text, video, voice, or music (e.g., what object is in the photo, what the content and emotion of the text are, what the content and emotion of the voice are, etc.). The deep neural network may include a convolutional neural network (CNN), a recurrent neural network (RNN), an autoencoder, a generative adversarial network (GAN), and a restricted Boltzmann machine (RBM), a deep belief network (DBN), a Q network, a U network, a Siamese network, a generative adversarial Network (GAN), etc. The description of the deep neural network described above is only an example and the present disclosure is not limited thereto.

In one embodiment of the present disclosure, the network function may include an autoencoder. The autoencoder may be a type of artificial neural network to output output data similar to input data. The autoencoder may include at least one hidden layer, and an odd number of hidden layers may be placed between the input and output layers. The number of nodes in each layer may be reduced from the number of nodes in the input layer to an intermediate layer called a bottleneck layer (encoding), and then expanded symmetrically from the bottleneck layer to the output layer (symmetrical to the input layer). The autoencoder may perform nonlinear dimensionality reduction. The number of input layers and output layers may correspond to the dimensionality after preprocessing of the input data. The autoencoder may have a structure in which the number of nodes in the hidden layer included in the encoder decreases as the distance from the input layer increases. If the number of nodes in the bottleneck layer (the layer with the fewest nodes located between the encoder and decoder) is too small, a sufficient amount of information may not be conveyed, so the number of nodes in the bottleneck layer may be kept above a certain number (e.g., more than half of the input layers, etc.).

The neural network may be trained by at least one of supervised learning, unsupervised learning, semi-supervised learning, or reinforcement learning. Learning of the neural network may be a process of applying knowledge for the neural network to perform a specific operation to the neural network.

The neural network may be trained to minimize output errors. The neural network training is a process of repeatedly inputting learning data into the neural network, calculating the output of the neural network and the error of the target for the learning data, and updating the weight of each node in the neural network by backpropagating the error of the neural network is transferred from the output layer of the neural network to the input layer in the direction of reducing the error. In the case of supervised learning, learning data in which the correct answer is labeled in each learning data is used (i.e., labeled learning data), and in the case of unsupervised learning, no correct answer may be labeled in each learning data. That is, for example, in the case of supervised learning for data classification, the learning data may be data in which each learning data is labeled with a category. The labeled learning data is input to the neural network, and the error can be calculated by comparing the output (category) of the neural network with the label of the learning data. As another example, in the case of unsupervised learning for data classification, the error can be calculated by comparing the input learning data with the neural network output. The calculated error is backpropagated in the reverse direction (i.e., from the output layer to the input layer) in the neural network, and the connection weight of each node of each layer in the neural network can be updated according to backpropagation. The amount of change in the connection weight of each node updated may be determined according to a learning rate. The calculation of the neural network on the input data and backpropagation of errors can constitute a learning cycle (epoch). The learning rate may be applied differently depending on the number of repetitions of the learning cycle of the neural network. For example, in the early stages of the neural network training, a high learning rate may be used to increase efficiency by allowing the neural network to quickly achieve a certain level of performance, and in the later stages of the training, a low learning rate may be used to increase accuracy.

In the training of the neural network, the learning data may generally be a subset of actual data (i.e., the data to be processed using the trained neural network), so there may be a learning cycle where the error on the learning data decreases but the error on the actual data increases. Overfitting is a phenomenon in which errors in actual data increase due to excessive learning on learning data. For example, a phenomenon in which a neural network that has learned to recognize a cat by showing it a yellow cat fails to recognize a cat when it sees a non-yellow cat, which is a type of overfitting.

The overfitting may lead to increased errors in machine learning algorithms. Various optimization methods may be used to prevent such overfitting. To prevent overfitting, methods such as increasing the learning data, regularization, dropout to disable some of the network nodes during the training process, and use of a batch normalization layer may be applied.

The questionnaire 160 may extract the question about the correlation factor having the highest weight from the questionnaire DB 170 and provide the question to the user terminal 200. The questionnaire 160 may extract the life pattern schedule of the user through general questions including the monthly age of the user, the problem to be solved, or the like, and provide the user with a customized life pattern schedule without data input through a question about the correlation factor having the highest weight derived from the artificial intelligence learning model. The questionnaire 160 may extract the question about the correlation factor having the highest weight among a plurality of questions stored in the questionnaire DB 170 and provide the extracted question to the user.

The recommendation unit 180 may recommend non-prescription medication through symptom analysis based on the large language model (LLM). The customized life pattern and non-prescription medication ingredients may be generated and provided to the user terminal based on the questionnaire result input from the user terminal 200. When a question is input, the LLM performs a document search by similarity and keyword and evaluates the document, and when the document evaluation is determined to be not useful, the LLM regenerates the question and re-performs the document search. Moreover, when the document evaluation passes, the LLM generates an answer based on the extracted document and performs evaluation about the answer, when the answer is determined to be not useful, the LLM regenerates the answer, and when the answer is useful, the answer may pass.

The control unit 190 may control each configuration of the operation server. The control unit is a processor including one or more cores, and may include a processor for data analysis and deep learning of a central processing unit (CPU), a general-purpose graphics processing unit (GPGPU), a tensor processing unit (TPU), and the like of a computing device. The processor may read a computer program stored in a storage space and perform data processing for learning according to one embodiment of the present disclosure. According to one embodiment of the present disclosure, the processor may perform operations for learning a neural network. The processor may perform calculations for learning a neural network, such as processing input data for learning in deep learning (DL), extracting features from input data, calculating errors, and updating weights of a neural network using backpropagation. At least one of the CPU, GPGPU, and TPU of the processor may process learning of a network function. For example, the CPU and GPGPU may together process learning of a network function and classification of data using a network function. In addition, in one embodiment of the present disclosure, the processors of multiple computing devices may be used together to process learning of network functions and data classification using network functions. In addition, the computer program executed in the computing device according to one embodiment of the present disclosure may be a CPU, GPGPU, or TPU executable program.

The control unit 190 may include a feeding/sleep information receiving module, a feeding/sleep information storage module, an infant information storage module, a feeding/sleep recommendation calculation module, a feeding/sleep recommendation transmission module, an automatic alarm control module, an AI question receiving module, an AI question database 120i, an AI question interpretation module 120j, and an AI answer transmission module, depending on the embodiment.

The feeding/sleep information receiving module may be configured to receive the feeding/sleep information of infants in real time from the user terminal. Moreover, the feeding/sleep information receiving module may be configured to store the feeding/sleep information in the feeding/sleep information storage module. The feeding/sleep information includes information on an actual feeding amount of the day, an actual feeding time, and an actual sleep time of the day. The feeding/sleep information is information that can accurately determine when and how much was fed and when and how much time was slept. The feeding/sleep information may be collected by the user.

The infant information storage module is configured to store information on a date of birth, a gender, a period of life, a daily weight, and a daily height of an infant. Information such as the date of birth and gender of the infant may be stored in advance by the user terminal, and the period of life may be automatically calculated and stored every day. In addition, the daily weight and daily height may be collected and received every day through the user terminal and stored in the infant information storage module.

The feeding/sleep recommendation calculation module may be configured to automatically calculate the currently required feeding/sleep recommendation based on the feeding/sleep information and infant information. The recommendation may be calculated by determining how many hours after one feeding the next feeding is recommended based on the actual feeding amount and actual feeding time on the day. In addition, the feeding amount or feeding time can be calculated by considering whether the infant is currently sleeping. In addition, in the case of sleep time, the recommended sleep amount may be calculated by comprehensively considering how much the infant slept that day and whether it is better to wake the infant up and feed when the infant is currently sleeping. In particular, for infants whose sleep patterns change during the day and night, the sleep recommendation may be provided by considering whether sleep should be induced or interrupted to restore the sleep pattern to normal. This feeding/sleep recommendation may vary depending on how many weeks old the baby is, and may also vary depending on whether it is appropriate depending on the infant's current weight or height and the period of birth.

The feeding/sleep recommendation calculation module may be configured to automatically adjust the feeding/sleep recommendation depending on whether a developmental status of the infant is within the normal range according to the developmental status.

The feeding/sleep recommendation transmission module may be configured to transmit the feeding/sleep recommendation according to the above algorithm to the user terminal in real time.

The automatic alarm control module may be configured to automatically generate and output a report on whether the achievement is made by setting the achievement conditions for each step for each step above and provide the report to the user terminal.

The AI question receiving module may be configured to receive the question of the user from the user terminal. The user terminal may be configured to receive the question of the user through a microphone or touch input. The AI question database may be configured to store the AI question received from the AI question receiving module.

The AI question database may be configured to link all feeding/sleep information about the current condition of the infant in question, the status of compliance of the recommendation, and all information about the life period, gender, weight, and height, or the like of the infant, to the AI question.

The AI question interpretation module may be configured to generate the AI answer by synthesizing the AI question stored in the AI question database and all feeding/sleep information, the status of compliance of the recommendation, the life period, gender, weight, height, or the like of the infant linked to the AI question. The AI answer transmission module may be configured to transmit the AI answer to the user terminal in real time.

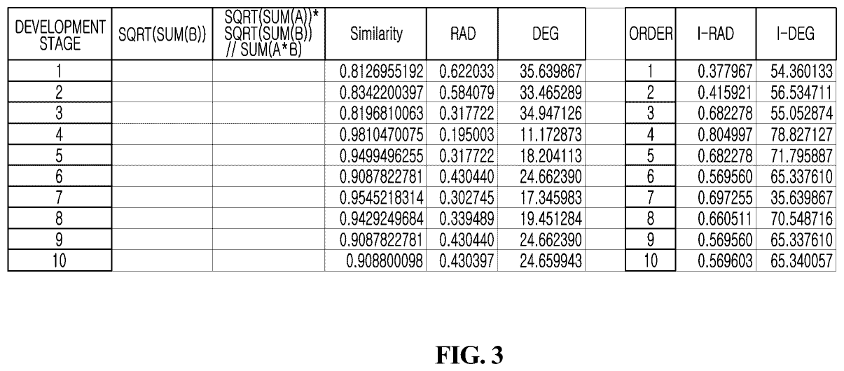

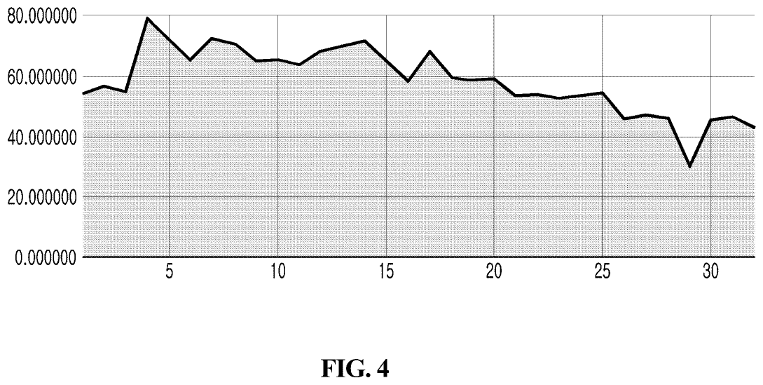

FIG. 3 and FIG. 4 are diagrams explaining a determination method using cosine similarity according to one embodiment of the present disclosure.

Referring to FIGS. 3 and 4, the similarity can be calculated according to the development stage by the monthly age using the actual input data for infants and toddlers and the cosine similarity for monthly age life patterns.

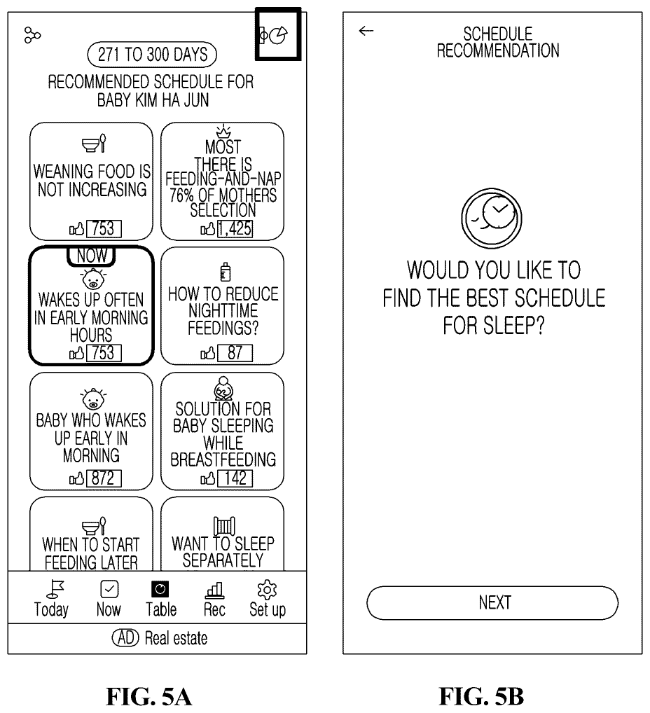

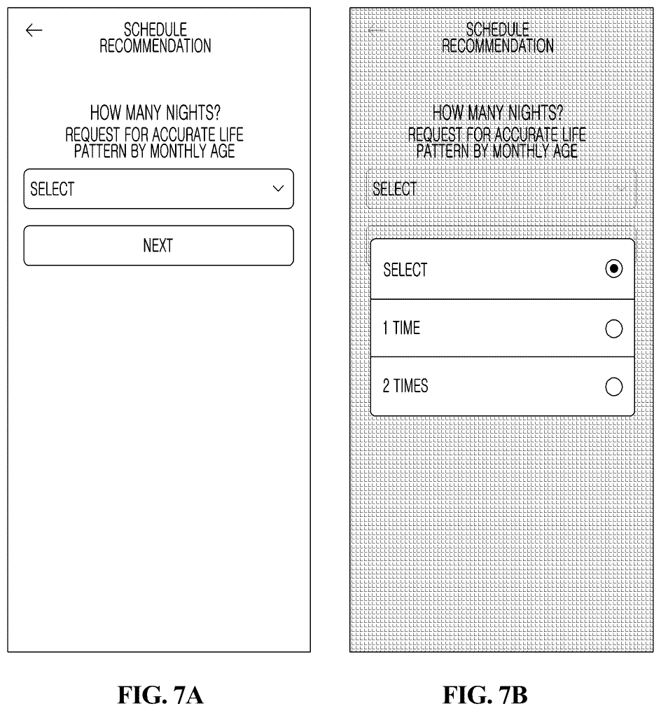

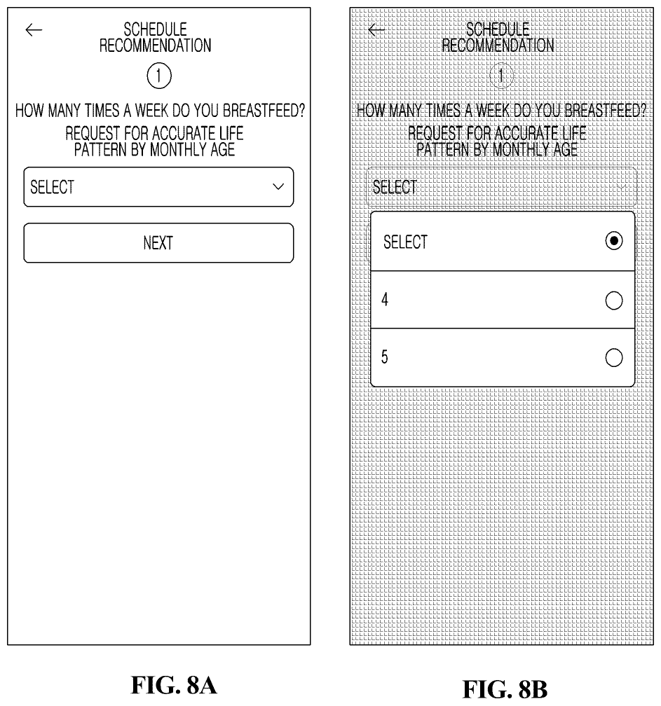

FIGS. 5A to 9C are diagrams explaining an application service operation screen according to one embodiment of the present disclosure.

Referring to FIGS. 5A to 9C, FIGS. 5A and 5B illustrate the application service screen operating on a user terminal, a screen (FIG. 5A) that selects a recommended schedule by the monthly age according to the characteristics of infants and toddlers may be displayed, and a screen (FIG. 5B) that can find and suggest an optimal schedule by selecting an icon on the upper right side of the screen may be provided.

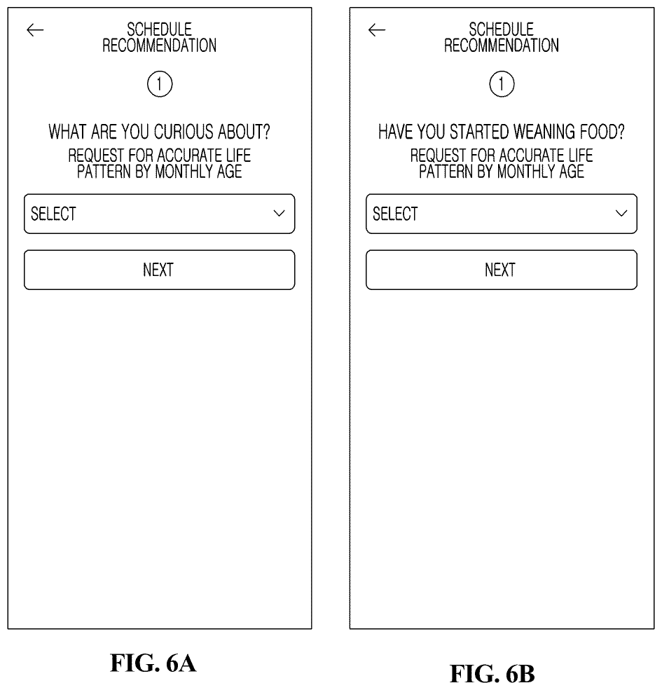

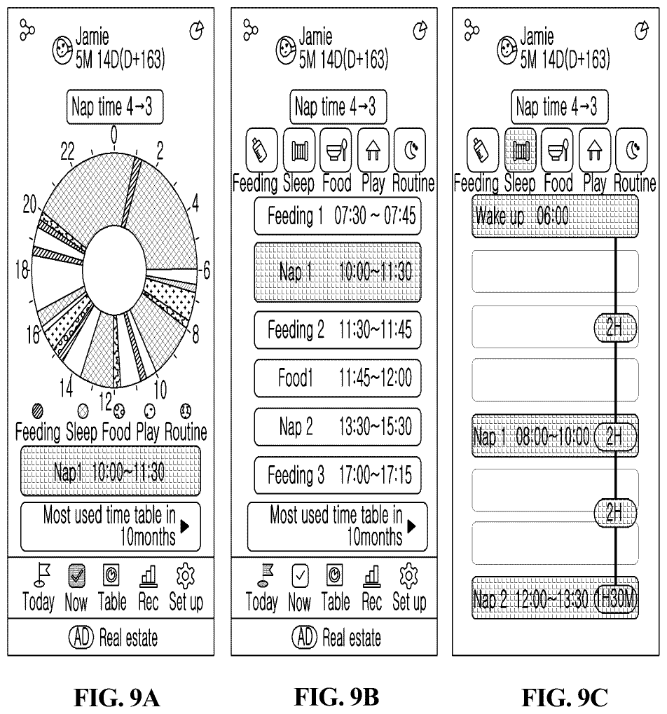

FIGS. 6A to 8B are screens in which the questionnaire extracts a question for the correlation factor having the highest weight from among a number of questions stored in the questionnaire DB and provides the extracted question to the user in order to suggest an optimal life pattern schedule. For example, questions such as “have you started weaning food?”, “how many times have you fed at night?”, and “how many times have you fed during the day?”, which are determined to be questions related to the correlation factor having the highest weight, may be asked, and answers may be input from the user terminal for the question. FIGS. 9A to 9C are screens illustrating the customized life pattern schedule generated by the recommendation unit based on answers received from the user terminal.

The present disclosure has been described with reference to the embodiments illustrated in the drawings, but the embodiments are merely exemplary, and those skilled in the art will understand that various modifications and other equivalent embodiments are possible from this. Therefore, the true technical protection scope of the present disclosure should be determined by the technical idea of the attached registration claims.

### DETAILED DESCRIPTION OF MAIN ELEMENTS

10: system for recommending AI-based solution for infant and toddler health care

| | |
|---|---|
| 100: operation server | 110: data collection unit |
| 120: life pattern extraction unit | 130: correlation factor extraction |
| 140: similarity measurement unit | 150: learning unit |
| 160: questionnaire | 170: questionnaire DB |
| 180: recommendation unit | 190: control unit |
| 200: user terminal | |

What is claimed is:

1. A method for recommending an artificial intelligence (AI)-based solution for infant and toddler health care, the method comprising:
collecting life pattern data including feeding data or sleep data of an infant and toddler, by a data collection unit;
refining development data and life pattern data from the collected life pattern data and extracting a plurality of monthly age life patterns by matching the refined life pattern data and development data, by a life pattern extraction unit;
analyzing the plurality of extracted monthly age life patterns to extract a problem and life pattern correlation factor for each monthly age life pattern, by a correlation factor extraction unit;
measuring a similarity between actual input data and the monthly age life pattern, by a similarity measurement unit;
inputting the actual input data and monthly age life patterns into an AI neural network model to perform learning, and deriving weight for each correlation factor, by a learning unit; and
recommending a non-prescription medication through symptom analysis based on a large language model (LLM), by a recommendation unit.

2. The method of claim 1, further comprising:
extracting a question about a correlation factor having the highest weight from a database and providing the extracted question to a user terminal, by a questionnaire; and
generating a customized life pattern and non-prescription medication ingredient based on a questionnaire result input from the user terminal and providing the generated customized life pattern and non-prescription medication ingredient to the user terminal, by the recommendation unit.

3. The method of claim 2, wherein the similarity measurement unit sets a variable of a standard lifestyle pattern for the infant and toddler by monthly age to A1 to A9, sets a variable of actual input data of a user to B1 to B9, and calculates a cosine similarity by applying a separate scale control table, the scale control table sets the weight to C1 to C9, and the variables C1 to C9 are weights according to expert knowledge and frequency of occurrence of major problems by monthly age, and the similarity is measured according to similarity calculation formulas below.
$$\begin{matrix}AA = SQRT [ {( A 1 * C 1 )}^{2} + {( A 2 * C 2 )}^{2} + … + {( A 9 * C 9 )}^{2} ] \\ BB = SQRT [ {( B 1 * C 1 )}^{2} + {( B 2 * C 2 )}^{2} + … + {( b 9 * C 9 )}^{2} ] \\ MM = [ ( A 1 * C 1 * B 1 * C 1 ) + ( A 2 * C 2 * B 2 * C 2 ) + … + ( A 9 * C 9 * B 9 * C 9 ) ] \\ Similarity = AA * BB / MM\end{matrix}$$

4. The method of claim 3, wherein the questionnaire extracts a life pattern schedule of the user through a general question including the monthly age of the user and a problem to be solved, and extracts a question about the correlation factor having the highest weight among a plurality of questions stored in a questionnaire DB and provides the extracted question to the user.

5. A system for recommending an artificial intelligence (AI)-based solution for infant and toddler health care, the system comprising:
an operation server; and
a user terminal,
wherein the operation server includes
a data collection unit configured to collect life pattern data including feeding data or sleep data of an infant and toddler,
a life pattern extraction unit configured to refine development data and life pattern data from the collected life pattern data and extract a plurality of monthly age life patterns by matching the refined life pattern data and development data, by a life pattern extraction unit,
a correlation factor extraction unit configured to analyze the plurality of extracted monthly age life patterns to extract a problem and life pattern correlation factor for each monthly age life pattern,
a similarity measurement unit configured to measure a similarity between actual input data and the monthly age life pattern,
a learning unit configured to input the actual input data and monthly age life patterns into an AI neural network model to perform learning and derive weight for each correlation factor,
a questionnaire configured to extract a question about a correlation factor having the highest weight from a database and providing the extracted question to the user terminal, and
a recommendation unit configured to generate a customized life pattern and non-prescription medication ingredient based on a questionnaire result input from the user terminal and provides the customized life pattern and non-prescription medication ingredient to the user terminal.
